(19) United States
(12) Patent Application Publication
(10) Pub. No.: US 2021/0401313 A1
(43) Pub. Date: Dec. 30, 2021

(54) Optimizing Sensor Pressure in Blood Pressure Measurements Using a Wearable Device

(71) Applicant: ChroniSense Medical Ltd., Yokneam (IL)

(72) Inventor: Daniel H. Lange, Kfar Vradim (IL)

(21) Appl. No.: 17/463,284

(22) Filed: Aug. 31, 2021

Related U.S. Application Data
(63) Continuation-in-part of application No. 15/226,881, filed on Aug. 2, 2016, now Pat. No. 11,160,461.
Application No. 15/226,881 is a continuation-in-part of application No. 14/738,666, filed on Jun. 12, 2015, now Pat. No. 11,160,459.
Application No. 14/738,666 is a continuation-in-part of application No. 14/738,636, filed on Jun. 12, 2015.
Application No. 14/738,636 is a continuation-in-part of application No. 14/738,711, filed on Jun. 12, 2015, now Pat. No. 10,470,692.

(51) Int. Cl. A61B 5/0225; A61B 5/021; A61B 5/024; A61B 5/318; A61B 5/00

(52) U.S. Cl. CPC A61B 5/02255; A61B 5/02125; A61B 5/02416; A61B 5/7405; A61B 5/318; A61B 5/746; A61B 5/7455; A61B 5/02438

(57) ABSTRACT

Systems and methods for optimizing sensor pressure in blood pressure (BP) measurements using a wearable device are provided. An example method includes recording photoplethysmogram (PPG) data using a PPG sensor of a wearable device while a pressure applied by the PPG sensor to a blood artery of a user is gradually increasing, monitoring a pulsating parameter associated with the PPG data, determining that the pulsating parameter has passed a critical value, in response to the determination, causing the increase of the pressure to stop, recording further PPG data using the PPG sensor and electrocardiogram (ECG) data using input plates of the wearable device, analyzing the further PPG data and the ECG data to determine a pulse transit time (PTT), a pulse rate (PR), and a diameter parameter, and determining, using a pre-defined model, a BP based on the PTT, the PR, and the diameter parameter.

### CROSS-REFERENCE TO RELATED APPLICATIONS

The present application is a Continuation in Part of U.S. patent application Ser. No. 15/226,881, titled “Blood Pressure Measurement Using a Wearable Device”, filed on Aug. 2, 2016. The application Ser. No. 15/226,881 is a Continuation in Part of U.S. patent application Ser. No. 14/738,666, titled “Monitoring Health Status of People Suffering from Chronic Diseases,” filed on Jun. 12, 2015, and is a Continuation in Part of U.S. patent application Ser. No. 14/738,636, titled “Wearable Device Electrocardiogram,” filed on Jun. 12, 2015, and is also a Continuation in Part of U.S. patent application Ser. No. 14/738,711, titled “Pulse Oximetry,” filed on Jun. 12, 2015. The disclosures of the aforementioned applications are incorporated herein by reference for all purposes.

### FIELD

The present application relates to systems and methods for monitoring the health status of people, and more specifically to systems and methods for optimizing sensor pressure in continuous or intermittent non-invasive blood pressure (NIBP) measurements using wearable devices.

### BACKGROUND

It should not be assumed that any of the approaches described in this section qualify as prior art merely by virtue of their inclusion in this section.

Blood pressure (BP) is one of the basic medical parameters used to diagnose human health condition. The most accurate methods for BP measurements involve insertion of a catheter into a human artery. However, the BP measurements using a catheter are invasive and costly since they require a medical professional to perform the measurements and, typically, can only be performed in a medical facility environment.

Less accurate methods for BP measurements include use of an inflatable cuff to pressurize a blood artery. There are numerous cuff-based portable devices for BP measurements that patients can use at home and do not require assistance of a medical professional. However, cuff-based measurements require inflation and deflation of the inflatable cuff. Therefore, such devices are cumbersome to use and not suitable for ongoing BP measurements.

Some cuff-less devices for BP measurements use an electrical sensor to measure an electrocardiogram (ECG) and optical sensors to measure a photoplethysmogram (PPG). The ECG and PPG can be analyzed to determine pulse transit time (PTT). Because the PTT is in-part inversely proportional to the BP, the BP can in some cases be determined from the PTT using a pre-defined relationship. However, changes in a cardio-vascular status of a patient require often re-calibration of PTT based blood pressure measurements. Cuff-less devices can potentially provide continuous monitoring of the BP while imposing a minimal burden on normal activities when worn on various body parts such as a finger, a wrist, or an ankle.

Determining the BP based on the PTT alone may not be sufficiently accurate because of other cardiovascular parameters affecting hemodynamics such as vascular resistance, cardiac output, pulse rate (PR), temperature of a finger (if PPG is measured at the finger), and so forth. To compensate for influences of other parameters, some existing techniques for measuring of BP using the PPG include applying correction factors to account for the vascular resistance and age of patient. The correction factors can be determined by an empirical formula. Some other techniques attempt to determine compensation factors to compensate for various additional influences (for example, contacting force to sensors, nervous activity and cardiac output of patient, and ambient temperature). The compensation factors can be determined using a calibration process.

However, all currently known methods for cuff-less, non-inflatable BP or NIBP monitoring require frequent re-calibration to compensate for unaccounted changes in the cardiovascular status of a patient. Moreover, in the PTT and BP measurements carried out using wearable devices, the accuracy of the PTT and BP depends on the pressure that sensors of the wearable device apply to the skin of patient and location of the sensors with respect to blood vessels of a patient. Because the pressure and the location of the sensors change each time the patient puts the wearable device on or corrects location of the wearable device on their body, the corresponding re-calibration would be also required to account for change in the pressure and the location of the sensors. Therefore, there is a need for an NIBP monitoring that can account for changes in the pressure and location of the sensors without frequent re-calibrations.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to one aspect of the present disclosure, systems and methods for optimizing sensor pressure in blood pressure measurements with a wearable device are provided. An example method may include recording, by at least one processor, photoplethysmogram (PPG) data using a PPG sensor of a wearable device while a pressure applied by the PPG sensor to a blood artery of a user is gradually increasing. The method may also include monitoring, by the processor, a pulsating parameter associated with the PPG data. The pulsating parameter may change in response to the gradually increasing pressure. The method may also include determining, by the processor, that the pulsating parameter has passed a critical value. In response to the determination, the method may stop the increase of the pressure in response to a command received from the processor.

The method may also include recording, by the processor, further PPG data using the PPG sensor and electrocardiogram (ECG) data using input plates of the wearable device. The method may then analyze, by the processor, the further PPG data and the ECG data to determine a pulse transit time (PTT), a pulse rate (PR), and a diameter parameter. The diameter parameter may include a change in the diameter of the blood artery. The method may determine, by the at least one processor and using a pre-defined model, a BP based on the PTT, the PR, and the diameter parameter. The pre-defined model can establish a relationship between the PTT, the PR, the diameter parameter, and the BP.

The wearable device may include a pressure applying device configured to gradually apply an external pressure to the PPG sensor.

The pressure can be increased by the user gradually applying an external pressure to the PPG sensor. The wearable device may include an alarm unit configured to prompt the user to stop applying the external pressure after the pulsating parameter has passed the critical value. The alarm unit may include a haptic device. The alarm unit may include a sound generating device.

The pulsating parameter may include a difference between a maximum and a minimum of the PPG data. The determination that the pulsating parameter has passed the critical value may include determining that the pulsating parameter has stopped increasing and started decreasing. Prior to recording the further PPG data, the processor may cause a decrease in the pressure to allow the pulsating parameter return to the maximum.

The determination of the diameter parameter may include modifying the further PPG data by removing, from the further PPG data, an additive contribution resulting from a reflection of a light signal from a surface of a skin covering the blood artery and near-surface tissues underlying the skin and covering the blood artery and keeping, in the PPG data, a contribution resulting from the reflection of the light signal from the blood artery unchanged. The additive contribution can be predetermined using a calibration process. The change in the diameter of the blood artery can be determined based on a ratio

$$\frac{A C}{D C} ,$$

wherein AC is an alternating current component of the modified PPG data, and DC is a direct current component of the modified PPG data. The blood artery can be a radial artery at a wrist.

According to another example embodiment of the present disclosure, the steps of the method for optimizing sensor pressure in blood pressure measurements using a wearable device are stored on a non-transitory machine-readable medium comprising instructions, which when implemented by one or more processors perform the recited steps.

Other example embodiments of the disclosure and aspects will become apparent from the following description taken in conjunction with the following drawings.

### BRIEF DESCRIPTION OF THE DRAWINGS

Embodiments are illustrated by way of example and not limitation in the figures of the accompanying drawings, in which like references indicate similar elements.

FIG. 1 is a block diagram showing an example system for performing a blood pressure measurement using a wearable device.

FIG. 2 is a block diagram showing components of an example device for performing blood pressure measurement.

FIG. 3 is block diagram illustrating an example device for measuring arterial blood pressure at a wrist.

FIG. 4 shows an example plot of an ECG and an example plot of a PPG.

FIG. 5 shows an example plot of a PPG and an example plot of a blood vessel diameter.

FIG. 6 is a flow chart showing an example method for performing blood pressure measurements.

FIG. 7 shows a diagrammatic representation of a computing device for a machine, within which a set of instructions for causing the machine to perform any one or more of the methodologies discussed herein can be executed.

FIG. 8A is diagrammatic representation of a blood vessel and optical sensor(s).

FIG. 8B is a plot of a compliance of a blood vessel, according to an example embodiment.

FIG. 9 shows plots of PPGs measured at different values of a sensor pressure, according to an example embodiment.

FIG. 10 is a flow chart showing an example method for optimizing sensor pressure in blood pressure measurements.

### DETAILED DESCRIPTION

The following detailed description includes references to the accompanying drawings, which form a part of the detailed description. The drawings show illustrations in accordance with exemplary embodiments. These exemplary embodiments, which are also referred to herein as “examples,” are described in enough detail to enable those skilled in the art to practice the present subject matter. The embodiments can be combined, other embodiments can be utilized, or structural, logical and electrical changes can be made without departing from the scope of what is claimed. The following detailed description is, therefore, not to be taken in a limiting sense, and the scope is defined by the appended claims and their equivalents.

The present disclosure provides systems and methods for performing BP measurement. Embodiments of the present disclosure allow for continuous or intermittent measuring of blood pressure of a patient in a non-intrusive manner while, for example, the patient is at home, at work, outdoors, traveling, or located at some other stationary or mobile environment. Embodiments of the present disclosure include a wearable device. The wearable device can be worn at a wrist, ankle, chest, neck, or positioned at other sites on a human body. The wearable device can allow measuring blood pressure of the patient without requiring the patient to take an active role in the process. The blood pressure data collected over an extended period of time can be analyzed to detect and track trends in medical parameters and to make conclusions concerning symptoms and progression of one or more chronic diseases from which the patient may suffer.

Some embodiments of the present disclosure can allow optimizing sensor pressure in BP measurements to increase accuracy of determination of the BP. According to some example embodiments, method for optimizing sensor pressure in BP measurements using a wearable deice may include recording, by at least one processor, photoplethysmogram (PPG) data using a PPG sensor of a wearable device while a pressure applied by the PPG sensor to a blood artery of a user is gradually increasing. The method may include monitoring, by the processor, a pulsating parameter associated with the PPG data. The pulsating parameter may change in response to the gradually increasing pressure. The method may include determining, by the processor, that the pulsating parameter has passed a critical value. In response to the determination, the method may include causing, by the processor, the increase of the pressure to stop. The method may include recording, by the processor, further PPG data using the PPG sensor and electrocardiogram (ECG) data using input plates of the wearable device. The method may include analyzing, by the processor, the further PPG data and the ECG data to determine a pulse transit time (PTT), a pulse rate (PR), and a diameter parameter. The diameter parameter may include a change in the diameter of the blood artery. The method may include determining, by the at least one processor and using a pre-defined model, a BP based on the PTT, the PR, and the diameter parameter. The pre-defined model can establish a relationship between the PTT, the PR, the diameter parameter, and the BP.

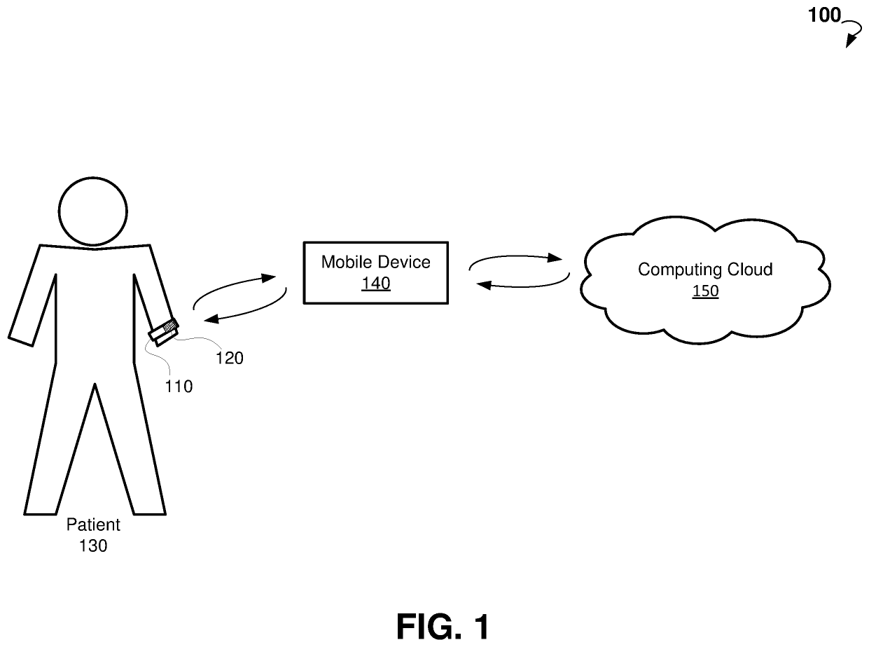

Referring now to FIG. 1, an example system 100 for performing blood pressure measurements is shown. The system 100 can include at least a wearable device 110. The wearable device 110 can include sensors 120. In some embodiments, the wearable device 110 is worn by a patient 130 (for example, on a wrist, ankle, earlobe, neck, chest, fingertip, and the like) for an extended period of time. In various embodiments, the wearable device 110 can be carried out as a watch, a bracelet, a wristband, a belt, a neck band, and the like.

The wearable device 110 can be operable to constantly collect, via sensors 120, sensor data from a patient 130. Based on the sensor data, the wearable device 110 can be operable to provide PPG and ECG. The PPG and ECG can be further used to obtain further medical parameters (for example, pulse rate, pulse transition time, blood pressure, and so forth).

In some embodiments, the system 100 includes a mobile device 140. The mobile device 140 can be communicatively coupled to the wearable device 110. In various embodiments, the mobile device 140 is operable to communicate with the wearable device 110 via a wireless connection such as, for example, Wi-Fi, Bluetooth, Infrared (IR), and the like. The mobile device 140 can include a mobile phone, a smart phone, a phablet, a tablet computer, a notebook, and so forth. The mobile device 140 can be operable to receive the sensor data and analyze the sensor data to provide ECG and PPG.

In further embodiments, the system 100 may include a cloud-based computing resource (also referred to as a computing cloud) 150. In some embodiments, the computing cloud 150 includes one or more server farms/clusters comprising a collection of computer servers and is co-located with network switches and/or routers. In certain embodiments, the mobile device 140 is communicatively coupled to the computing cloud 150. The mobile device 140 can be operable to send the sensor data to the computing cloud 150 for further analysis (for example, for extracting medical parameters from the ECG and PPG and storing the results). The computing cloud 150 can be operable to run one or more applications and to provide reports regarding a health status of the patient, based on trends in medical parameters over time.

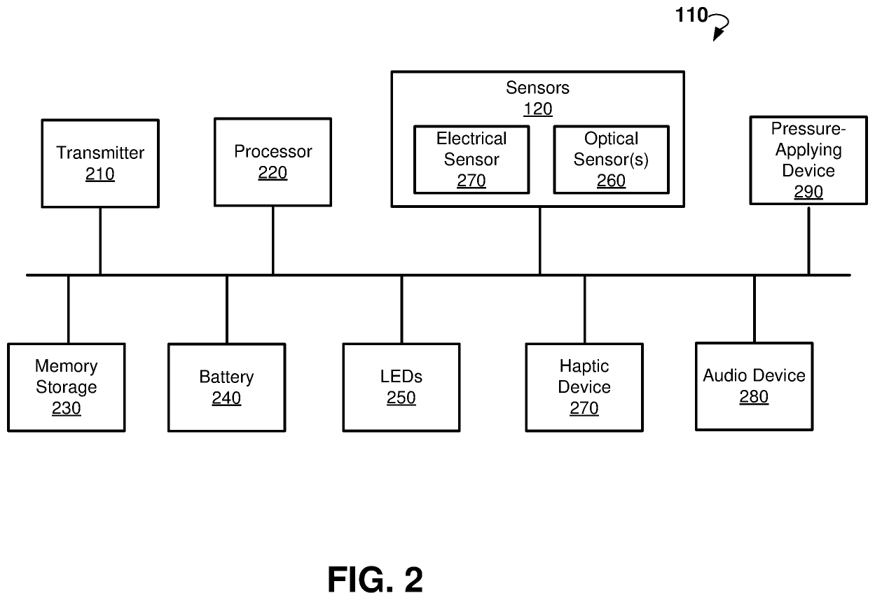

FIG. 2 is a block diagram illustrating components of wearable device 110, according to an example embodiment. The example wearable device 110 includes a transmitter 210, a processor 220, memory storage 230, a battery 240, light-emitting diodes (LEDs) 250, optical sensor(s) 260, electrical sensor 270, a haptic device 270, an audio device 280, and a pressure-applying device 290. The wearable device 110 may comprise additional or different components to provide a particular operation or functionality. Similarly, in other embodiments, the wearable device 110 includes fewer components that perform similar or equivalent functions to those depicted in FIG. 2.

The transmitter 210 can be configured to communicate with a network such as the Internet, a Wide Area Network (WAN), a Local Area Network (LAN), a cellular network, and so forth, to send data streams (for example sensor data, PPG data, and messages).

The processor 220 can include hardware and/or software, which is operable to execute computer programs stored in memory 230. The processor 220 can use floating point operations, complex operations, and other operations, including processing and analyzing data obtained from electrical sensor 270 and optical sensor(s) 260.

In some embodiments, the battery 240 is operable to provide electrical power for operation of other components of the wearable device 110. In some embodiments, the battery 240 is a rechargeable battery. In certain embodiments, the battery 240 is recharged using an inductive charging technology.

In various embodiments, the LEDs 250 are operable to emit light signals. The light signals can be of a red wavelength (typically 660 nm) or infrared wavelength (660 nm). Each of the LEDs 250 is activated separately and accompanied by a “dark” period where neither of the LEDs 250 is on to obtain ambient light levels. In some embodiments, a single LED 250 can be used to emit both the infrared and red light signals. The lights can be absorbed by human blood (mostly by hemoglobin). The oxygenated hemoglobin absorbs more infrared light while deoxygenated hemoglobin absorbs more red light. Oxygenated hemoglobin allows more red light to pass through while deoxygenated hemoglobin allows more infrared light to pass through. In some embodiments of the present disclosure, the LEDs 250 are also operable to emit light signals of isosbestic wavelengths (typically 810 nm and 520 nm). Both oxygenated hemoglobin and deoxygenated hemoglobin absorb the light of the isosbestic wavelengths equally.

The optical sensor(s) 260 (typically a photodiode) can receive light signals modulated by human tissue. Intensity of the modulated light signal represents a PPG. Based on the changes in the intensities of the modulated light signals, one or more medical parameters, such as, for example, oxygen saturation, arterial blood flow, pulse rate, and respiration, can be determined.

The LEDs 250 and optical sensor(s) 260 can be utilized in either a transmission or a reflectance mode for pulse oximetry. In the transmission mode, the LEDs 250 and optical sensor(s) 260 are typically attached or clipped to a translucent body part (e.g., a finger, toe, and earlobe). The LEDs 250 are located on one side of the body part while the optical sensor(s) 260 are located directly on the opposite site. The light passes through the entirety of the body part, from one side to the other, and is thus modulated by the pulsating arterial blood flow. In the reflectance mode, the LEDs 250 and optical sensor(s) 260 are located on the same side of the body part (e.g. a forehead, a finger, and a wrist), and the light is reflected from the skin and underlying near-surface tissues back to the optical sensor(s) 260.

The haptic device 270 can be configured to provide the patient a haptic feedback. For example, the haptic device may include a tap-in device, to apply a force or vibration to skin of the patient.

The audio device 280 can be configured to provide the patient a sound feedback. The audio device 280 can include a beeper configured to generate sounds of one or more pre-determined wavelengths.

The pressure-applying device 290 may be configured to apply external pressure to the optical sensor(s) 260 to force the optical sensor(s) 260 to contact the skin of a patient with different values of a contact force. In some embodiments, the pressure-applying device 290 may include an electrical motor and a spring touching the optical sensor(s) 260. The electrical motor can be configured to stretch the spring gradually causing the optical sensor(s) 260 to apply gradually increasing pressure to the skin of the patient. In other embodiments, the pressure-applying device 290 can include an electrical pump and an inflatable cuff configured to generate external pressure against the optical sensor(s) 260. The electrical pump may inflate the cuff gradually causing the optical sensor(s) 260 to apply gradually increasing pressure to the skin of the patient.

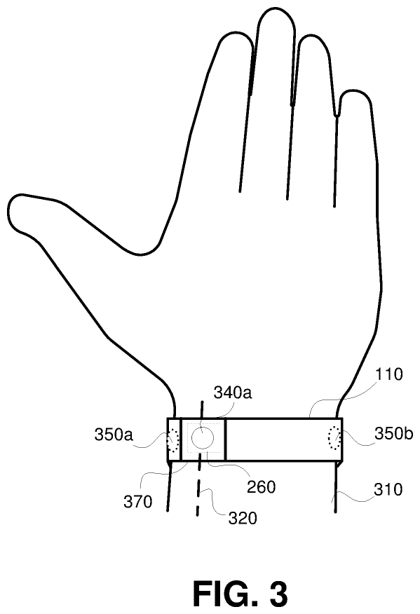

FIG. 3 is a block diagram illustrating an example wearable device 110 placed around a wrist of a patient. In the example of FIG. 3, the wearable device 110 is carried out in a shape of a watch, a ring, and/or a bracelet.

The electrical sensor 270 can include a differential amplifier operable to measure the electrical signal from the wrist. The electrical sensor 270 can include two or more active amplifier input plates embedded in the wearable device at opposite ends. For example, input plates 350a and 350b can be placed in contact with, respectively, the left and right sides of the wrist 310. Alternatively or additionally, two input plates can be placed on opposite sides of the wearable device 110. In some embodiments, the first input plate 340a can be placed on the outer side the wearable device. The second input plate can be placed on the inner side of the wearable device. The second input plate can be in contact with the skin of the patient when the patient wears the wearable device. In some embodiments, the first input plate 340a can be placed in an area 370 of the wearable device 110. The area 270 may cover the radial artery 320 of a patient. The optical sensor(s) 260 can be placed on inner side of the area 370 of the wearable device 110.

In some embodiments, the optical sensor(s) 260 can be placed beneath a pulsating artery travelling along the arm and into a wrist 310. In some embodiments, a radial artery 320 passing in the inner wrist is used for measurements by the optical sensor(s) 260. In other embodiments, other arteries such as the ulnar artery, may be used. An external light source generating constant lighting can be used to radiate the pulsating artery. A beam reflected from the pulsating artery can be intercepted by the optical sensor(s) 260. In certain embodiments, a light of isosbestic wavelength is used to radiate the pulsating artery.

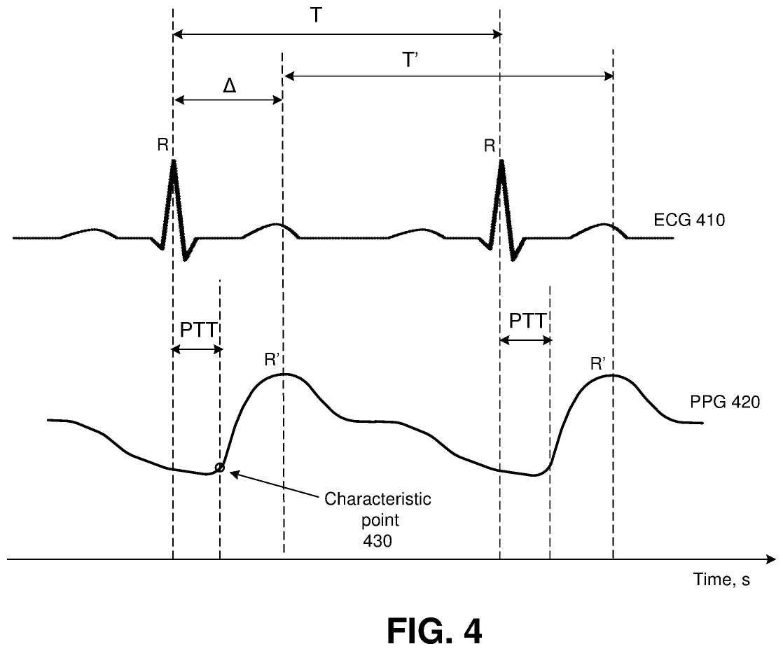

FIG. 4 shows plots of an example an example plot of an ECG 410, and an example plot of a PPG 420. The ECG 410 can be recorded with electrical sensor 270 using input plates placed on the wearable device 110. The ECG 410 can include R peaks corresponding to heart beats. Taking measurements from a single hand or a single wrist is challenging because the difference in voltages between measured locations is miniscule. The electrical signal measured at the wrist can include an ECG 410 and a noise. The noise can be caused by muscle activity, patient movements, and so forth. The noise component can be larger than the ECG. In some embodiments, the signal-to-noise ratio (SNR) is in the range of −40dB to −60dB. An example method for measuring a “clean” ECG from a wrist is described in U.S. patent application Ser. No. 14/738,666, titled “Wearable Device Electrocardiogram,” filed on Jun. 12, 2015.

The PPG 420 can be obtained by sensing a change in the color of skin. The change of the skin color is caused by a blood flow in a pulsating artery. In some embodiments, the PPG 420 can include peaks R′ related to the heart beats. Since it takes a time for blood to flow from the heart to the wrist, the peaks R′ are shifted by time periods Δ relative to the heart beats R in ECG 420. In some embodiments, shifts Δ can be measured as shift of a waveform of PPG (complex of PPG corresponding to period T′ in FIG. 4) relative to a waveform of ECG (complex of ECG corresponding to period T in FIG. 4).

In various embodiments, ECG 410 and PPG 420 are used to estimate a PTT. In some embodiments, PTT is defined as a time interval between the R peak in ECG 410 and characteristic point 430 located at the bottom of the PPG 420. PTT is a parameter which inversely correlates to BP. PTT decreases as BP increases and PTT increases as BP decreases. Therefore, PTT can be used to estimate BP. In some embodiments, a regression equation can be derived to establish a relation between PTT and BP. The regression equation can be established for both systolic BP and diastolic BP. Alternatively in other embodiments, other mathematical models, such as neural networks, may be used to establish the relation between the PTT and BP.

The location of characteristic point 430 can be uncertain or hard to detect. For example, a shape of PPG at a foot can be diffused when a pulse rate is high. Therefore, in some embodiments, when location of characteristic point 430 is uncertain or hard to detect, shifts between specific features of the ECG and PPG (such as certain landmarks or peaks) corresponding to the same heartbeat are used as an estimate for PTT. In certain embodiments, PTT is estimated based on shifts between waveforms of ECG and PPG corresponding to the same heartbeat.

PTT depends on the shape and cross-section area of a blood vessel (for example, a pulsating artery at which measurement is performed) since speed of blood travelling through the blood vessel depends on the cross-section area of the blood vessel and blood pressure.

According to various embodiments of the present disclosure, ECG and PPG are used to estimate PTT, PR, and diameter of the blood vessel or a change in the diameter of the blood vessel. In some embodiments, PTT, PR, and the diameter of the blood vessel or the change in the diameter of the blood vessel are then used to estimate BP. In some embodiments, PTT is determined based on ECG and PPG. PR can be found using a time period between two consecutive peaks in ECG or two consecutive peaks in PPG. In some embodiments, the diameter of the blood vessel or the change in the diameter of the blood vessel can be estimated using PPG.

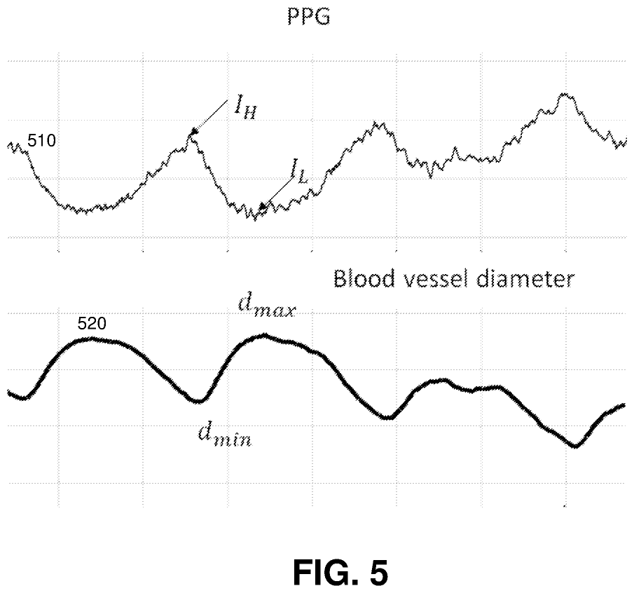

FIG. 5 shows an example plot of PPG 510 and an example plot of blood vessel diameter 520. The PPG 510 represents the intensity I of the light signal as modulated by a human tissue mostly due to a blood flow in the blood vessel. The high peaks (maximums) I_{H} of PPG 510 correspond to the low peaks dₘᵢₙ of the blood vessel diameter 520, and the low peaks I_{L} of the PPG 510 correspond to the high peaks dₘₐₓ of the blood vessel diameter 520.

In some embodiments, the detected PPG signal I, which is the intensity of light signal reflected from pulsating tissue, is modeled as follows:

I(t)=I₀*F*e^{-c*d(t)} (1).

In formula (1), I₀ represents an incident light intensity, F is indicates the absorption by pulsatile tissue, d(t) represents (arterial) blood vessel diameter, and c is overall absorption coefficient of blood hemoglobin derived from a mixture of both oxygen-saturated and non-oxygen saturated hemoglobin. Each of oxygen-saturated and non-oxygen saturated hemoglobin has its own particular value of absorption coefficient c for a particular wavelength of emitted light. Therefore, according to some embodiments, a light of isosbestic wavelength is used to radiate the pulsatile tissue allowing absorption coefficient c so it remains constant and independent of SpO2 oxygen saturation. The light absorption at the isosbestic wavelength is independent of SpO2 oxygen saturation because when a light of an isosbestic wavelength is used, the reflection from the oxygenized blood is the same as reflection from the non-oxygenized blood. In some embodiments, the isosbestic wavelength includes a near infrared wavelength 810 nm (NIR) and a green wavelength 520 nm (green). The NIR wavelength is more suitable for deeper vessels as it has deeper penetration while the green wavelength is more suitable for shallow vessels.

As shown in FIG. 5, the blood vessel diameter 520 changes periodically with the rhythm of the heart rate. The low peaks of the blood vessel diameter dₘᵢₙ correspond to the minimums of the absorption of the light by the blood and the high peaks of the light intensity I_{H}. The high peaks of the blood vessel diameter dₘₐₓ correspond to maximum absorption of the light by blood and the lowest peaks of the light intensity I_{L}. In some embodiments, the low peaks of the blood vessel diameter dₘᵢₙ can be considered to be constant as they reflect lowest diastole. The high peaks of the blood vessel diameter dₘₐₓ may vary relatively slowly due to, for example, fluctuations of blood pressure.

In some embodiments, it can be assumed that

I(t)≈I₀*F*(1−c*d(t)) (2).

Denoting further direct current (DC) component of PPG

DC=I₀*F (3)

and alternative current (AC) component

AC=I₀*F*c*d(t) (4),

an equation for determining blood vessel diameter d(t) can be written as:

$$( \frac{A c}{D c} ) = c * d ( t ) . \qquad ( 5 )$$

In equation (5), the AC component and DC component are found from PPG and absorption coefficient c is known. In some embodiments, change d(t)ₘₐₓ−d(t)ₘᵢₙ is used to estimate BP.

In other embodiments, BP is calculated from measured PTT, PR, and the diameter of the blood vessel or a change thereof using a pre-defined model. The pre-defined model describes a relationship between PTT, PR, and the diameter of the blood vessel and BP. In some embodiments, the pre-defined model is determined using statistical data collected during a calibration process. During the calibration process, a patient can wear the wearable device 110 to measure PTT, PR, and the diameter of the blood vessel or a change in the diameter of the blood vessel. Simultaneously, BP can be measured using an external device (for example, a conventional device for BP measurement). The calibration can be performed once at first usage of the wearable device 110 by a particular patient, and requires at least a single simultaneous measurement by the wearable device 110 and the external device. In other embodiments, several simultaneous measurements should be made to calibrate the wearable device 110 in a range of blood pressure values. The range of blood pressure values can be achieved by taking measurements at either or all the following: different times (hours of a day), different physical states of a patient, and different emotional states of the patient. Alternatively, lowering or elevating the arm and taking local blood pressure at the wrist with both an external device and the wearable device 110 can provide an effective means for mapping the PTT, PR, and diameter of the blood vessel or a change in the diameter of the blood vessel to a wide range of blood pressure values.

In some embodiments, the pre-defined model includes a three-dimensional model, wherein PTT, PR and the diameter of the blood vessel or a change in the diameter of the blood vessel are explanatory variables and systolic blood pressure is a dependent variable. Similarly, another three-dimensional model can be used to establish mathematical relationships between PTT, PR and diameter of blood vessel or a change in the diameter of the blood vessel as explanatory variables and diastolic blood pressure as a dependent variable.

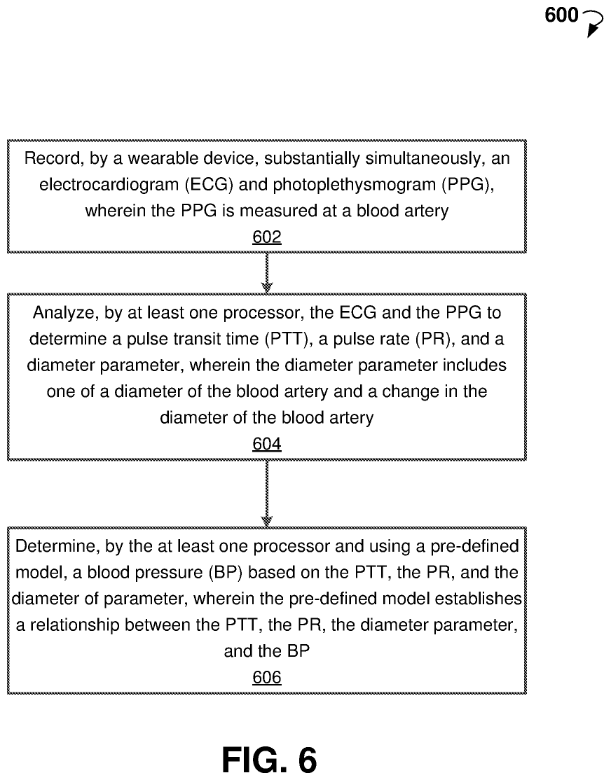

FIG. 6 is a flow chart showing steps of a method 600 for performing BP measurement, according to some embodiments. The method 600 can be implemented using wearable device 110 described in FIGS. 2 and 3 and system 100 described in FIG. 1. The method 600 may commence in block 602 with substantially simultaneous recording, by a wearable device, an ECG and a PPG. In some embodiments, PPG is measured at a blood artery. In some embodiments, ECG and PPG are recorded at a wrist.

In block 604, the method 600 proceeds with analyzing ECG and PPG to determine a PTT, a PR, and a diameter parameter. The diameter parameter may include a diameter of the blood artery or a change in the diameter of the blood artery. In block 606, the method 600 determines, based on PTT, PR, and the diameter parameter, BP using a pre-defined model. The pre-defined model establishes a relationship between the PTT, the PR, the diameter parameter, and the BP. In some embodiments, analysis of ECG and PPG and determination of PTT, the PR, the diameter parameter, and BP is performed locally using processor of the wearable device. In other embodiments, analysis of ECG and PPG and determination of PTT, the PR, the diameter parameter, and BP can be carried out remotely by a mobile device connected to the wearable device or in a computing cloud.

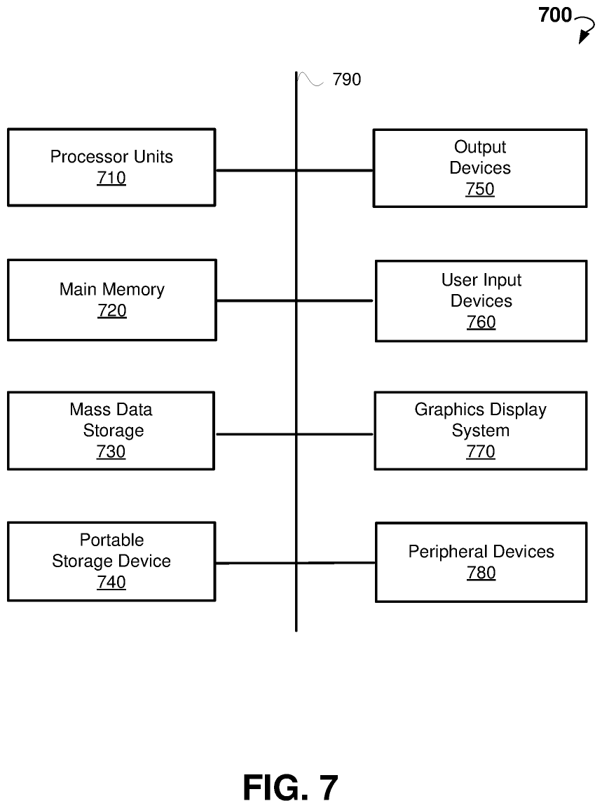

FIG. 7 illustrates a computer system 700 that may be used to implement embodiments of the present disclosure, according to an example embodiment. The computer system 700 may serve as a computing device for a machine, within which a set of instructions for causing the machine to perform any one or more of the methodologies discussed herein can be executed. The computer system 700 can be implemented in the contexts of the likes of computing systems, networks, servers, or combinations thereof. The computer system 700 includes one or more processor units 710 and main memory 720. Main memory 720 stores, in part, instructions and data for execution by processor units 710. Main memory 720 stores the executable code when in operation. The computer system 700 further includes a mass data storage 730, a portable storage device 740, output devices 750, user input devices 760, a graphics display system 770, and peripheral devices 780. The methods may be implemented in software that is cloud-based.

The components shown in FIG. 7 are depicted as being connected via a single bus 790. The components may be connected through one or more data transport means. Processor units 710 and main memory 720 are connected via a local microprocessor bus, and mass data storage 730, peripheral devices 780, the portable storage device 740, and graphics display system 770 are connected via one or more I/O buses.

Mass data storage 730, which can be implemented with a magnetic disk drive, solid state drive, or an optical disk drive, is a non-volatile storage device for storing data and instructions for use by processor units 710. Mass data storage 730 stores the system software for implementing embodiments of the present disclosure for purposes of loading that software into main memory 720.

The portable storage device 740 operates in conjunction with a portable non-volatile storage medium, such as a floppy disk, compact disk (CD), Digital Versatile Disc (DVD), or USB storage device, to input and output data and code to and from the computer system 700. The system software for implementing embodiments of the present disclosure is stored on such a portable medium and input to the computer system 700 via the portable storage device 740.

User input devices 760 provide a portion of a user interface. User input devices 760 include one or more microphones, an alphanumeric keypad, such as a keyboard, for inputting alphanumeric and other information, or a pointing device, such as a mouse, a trackball, stylus, or cursor direction keys. User input devices 760 can also include a touchscreen. Additionally, the computer system 700 includes output devices 750. Suitable output devices include speakers, printers, network interfaces, and monitors.

Graphics display system 770 includes a liquid crystal display or other suitable display device. Graphics display system 770 receives textual and graphical information and processes the information for output to the display device. Peripheral devices 780 may include any type of computer support device to add additional functionality to the computer system.

The components provided in the computer system 700 of FIG. 7 are those typically found in computer systems that may be suitable for use with embodiments of the present disclosure and are intended to represent a broad category of such computer components that are well known in the art. Thus, the computer system 700 can be a personal computer, handheld computing system, telephone, mobile computing system, workstation, tablet, phablet, mobile phone, server, minicomputer, mainframe computer, or any other computing system. The computer may also include different bus configurations, networked platforms, multi-processor platforms, and the like. Various operating systems may be used including UNIX, LINUX, WINDOWS, MAC OS, PALM OS, ANDROID, IOS, QNX, TIZEN and other suitable operating systems.

It is noteworthy that any hardware platform suitable for performing the processing described herein is suitable for use with the embodiments provided herein. Computer-readable storage media refer to any medium or media that participate in providing instructions to a central processing unit, a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD Read Only Memory disk, DVD, Blu-ray disc, any other optical storage medium, RAM, Programmable Read-Only Memory, Erasable Programmable Read-Only Memory, Electronically Erasable Programmable Read-Only Memory, flash memory, and/or any other memory chip, module, or cartridge.

In some embodiments, the computer system 700 may be implemented as a cloud-based computing environment, such as a virtual machine operating within a computing cloud. In other embodiments, the computer system 700 may itself include a cloud-based computing environment, where the functionalities of the computer system 700 are executed in a distributed fashion. Thus, the computer system 700, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computer system 700, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

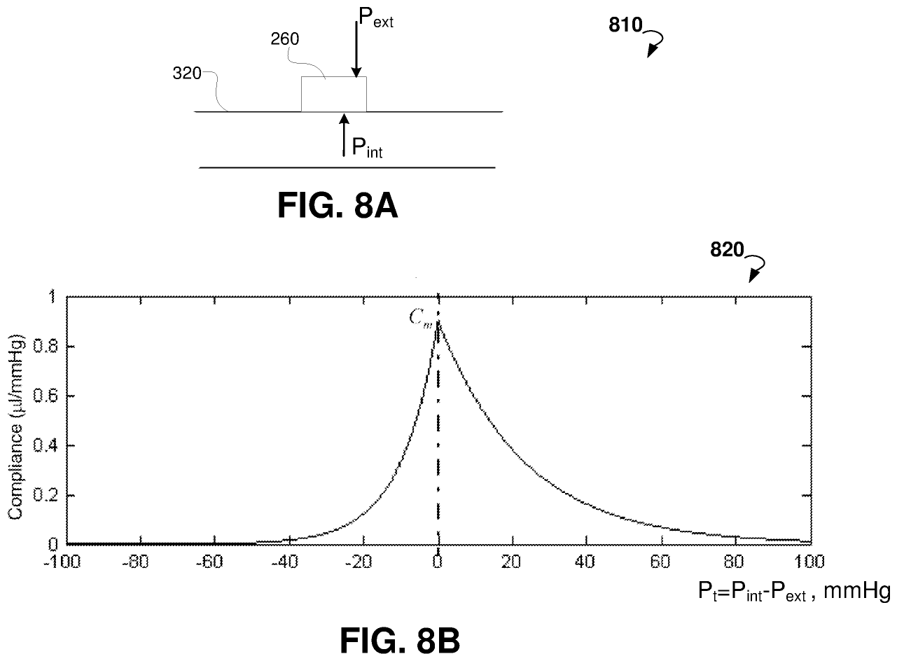

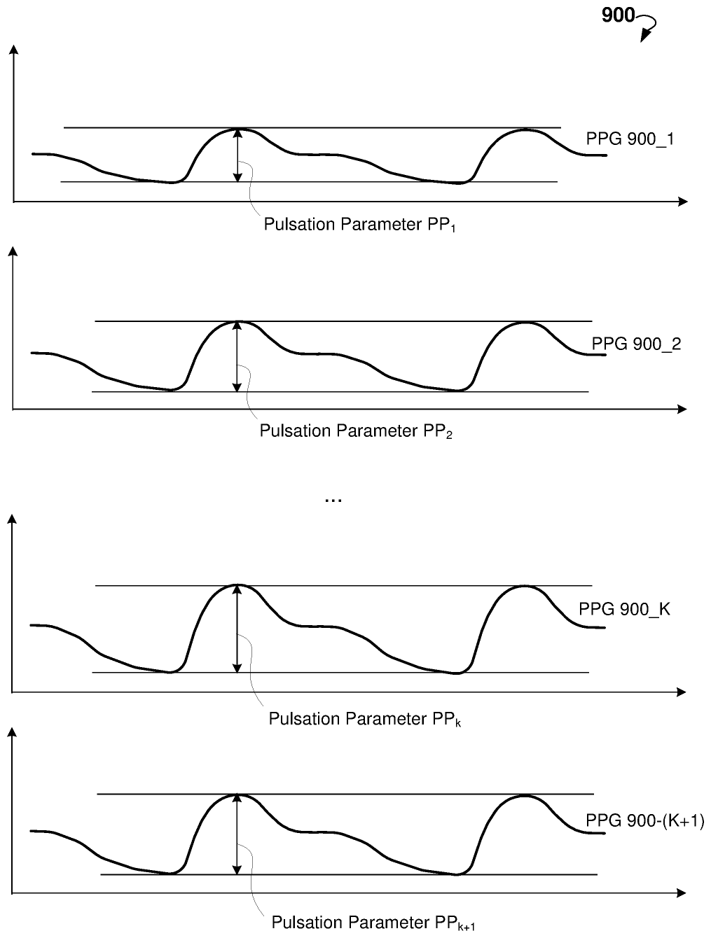

FIG. 8A is diagrammatic representation of a blood vessel 320 and optical sensor(s) 260. The optical sensor(s) 260 applies pressure Pₑₓₜ to the blood vessel 320. Pᵢₙₜ denotes mean intra-arterial pressure in the blood vessel 320. Determination of the PTT and the BP depend on the accuracy of determination of fluctuation Δd(t) of the blood vessel diameter d(t). The accuracy of determination of fluctuation Δd(t) of the blood vessel diameter d(t) can be contaminated due to either excessive or insufficient amount of the external pressure Pₑₓₜ applied to the blood vessel by the optical sensor(s) 260. Some values of external pressure Pₑₓₜ applied to the blood vessel may result in up to 5% error in PTT and up to 10% in BP.

The fluctuation of the Δd(t) of the blood vessel diameter d(t) depends on the properties of the blood vessel, specifically on compliance C. Compliance C can be determined as follows:

$$C = \frac{Δ V}{Δ P} , \qquad ( 6 )$$

where ΔV is the change of local volume of the blood vessel in response to change ΔP of distending pressure.

FIG. 8B is a plot of compliance C of a blood vessel. The value of the compliance C depends on value of transmural pressure Pₜ. The transmural pressure Pₜ is defined as a difference between the mean intra-arterial pressure Pᵢₙₜ and the external pressure Pₑₓₜ:

Pₜ=Pᵢₙₜ−Pₑₓₜ (7).

As shown in FIG. 8B, the compliance C reaches a maximum value at Pₜ=0. At the maximum value of compliance C the fluctuation of blood vessel volume ΔV (and, correspondently, the fluctuation Δd(t) of the blood vessel diameter d(t)) is maximum. If the external pressure Pₑₓₜ exceeds the mean intra-arterial pressure Pᵢₙₜ or the external pressure Pₑₓₜ is less than the mean intra-arterial pressure Pᵢₙₜ, then the compliance C is not maximum. In these situations, the fluctuation of blood vessel volume ΔV is not maximum.

As shown in FIG. 5, the fluctuation of the PPG 510 correlates with the fluctuation of the blood vessel diameter. Accordingly, at the maximum value of the compliance C, the fluctuation of the PPG 510 is also maximum. This fact can be used to determine a value of sensor pressure corresponding to the maximum value of the compliance C of the blood vessel.

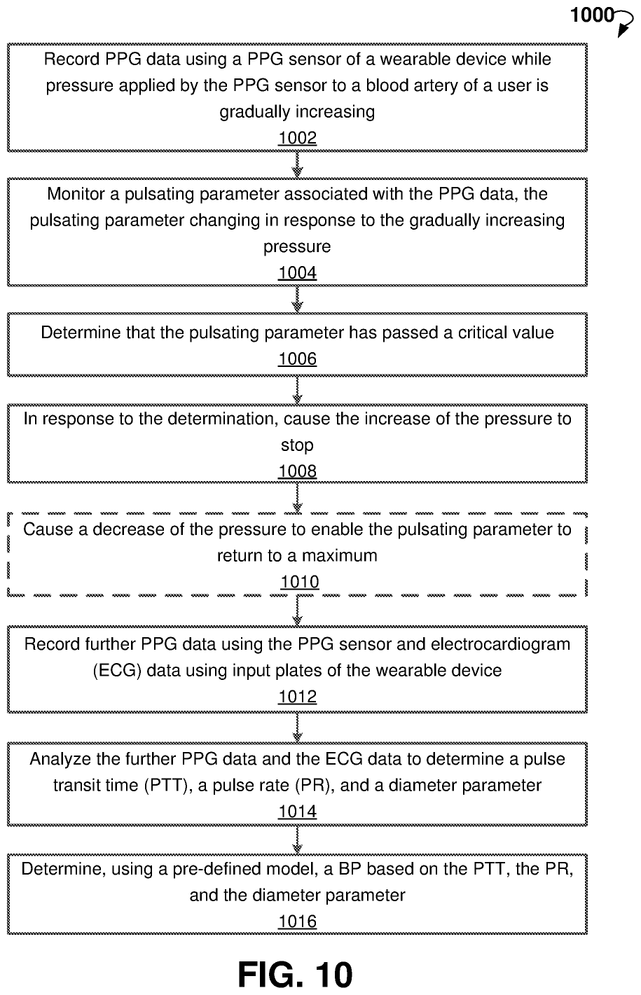

FIG. 9 shows plots of PPGs 900_k measured at different values P_{ext_k} of a pressure of an optical sensor(s) 260, according to some example embodiments. In these embodiments, the different values P_{ext_k} of the pressure applied by the optical sensor(s) 260 to blood vessel can be applied manually by a patient. The patient can be prompted to gradually apply pressure to the optical sensor(s) 260 by using a finger of the other hand at the area 370 of the wearable device 110. As shown in FIG. 3 the area 370 may cover the blood vessel on the wrist of the patient, for example, the radial artery 320. In some embodiment, the patient can be instructed to touch the input plate 340a of the electrical sensor 270 to allow recording two-hand ECG at the same time.

The processor of the wearable device 110 may record, using the optical sensor(s) 260, the PPGs 900_k. For each of the PPGs 900_k, the processor can determine a pulsation parameter PPk. In some embodiments, the pulsation parameter PPk can include a difference between maximums and minimums of the PPGs 900_k. The processor of the wearable device 110 may monitor the change of the pulsation parameter PPk while the pressure P_{ext_k} increases gradually. The processor may determine that the pulsation parameter PPk has passed a critical value, for example, a maximum. For example, the processor may determine that the pulsating parameter has stopped increasing and started decreasing. Correspondently, after the pulsation parameter PPk has passed the critical value, the processor may instruct the patient to stop increasing the pressure on the optical sensor(s) 260. For example, the processor may cause the haptic device 270 to apply a force or vibration to the skin of the patient. Alternatively, the processor may cause the audio device 280 to generate a sound. In some embodiments, the patient may start decreasing the pressure on the optical sensor(s) 260 to allow the pulsating parameter to return to the maximum. The processor may determine that the pulsating parameter has returned to the maximum and instruct the patient to stop decreasing the pressure. For example, the processor may cause the haptic device 270 to vibrate the skin of the patient. The pattern of such vibration can be different from the pattern of the vibration used to prompt the patient to stop increasing the pressure. Alternatively, the processor may cause the audio device 280 to generate a sound. The frequency of the sound can be different from the frequency of the sound used to prompt the patient to stop increasing the pressure.

In other embodiments, the different values P_{ext_k} of the pressure applied by the optical sensor(s) 260 to blood vessel can be created automatically by the pressure-applying device 290. The processor may cause the pressure-applying device 290 to stop the increase in the pressure after the pulsating parameter has passed the critical value. For example, the processor may cause the pressure-applying device 290 to stop the increase in the pressure after determining that the pulsating parameter has stopped increasing and started decreasing, that the pulsating parameter has passed the maximum. The processor may cause the pressure-applying device 290 to decrease the pressure applied by the optical sensor(s) 260 to blood vessel to allow the pulsating parameter to return to the maximum.

After determining that the pulsating parameter has returned to the maximum, the processor may proceed with blood pressure measurements using, for example, method 600 described above with reference to FIG. 6. At these conditions, compliance C of the blood vessel is maximum. Accordingly, the fluctuation of blood vessel volume ΔV and, correspondently, the fluctuation Δd(t) of the blood vessel diameter d(t)) is maximum. Therefore, the errors in estimates of diameter parameter (d(t)ₘₐₓ−d(t)ₘᵢₙ), BP, and PTT are minimum.

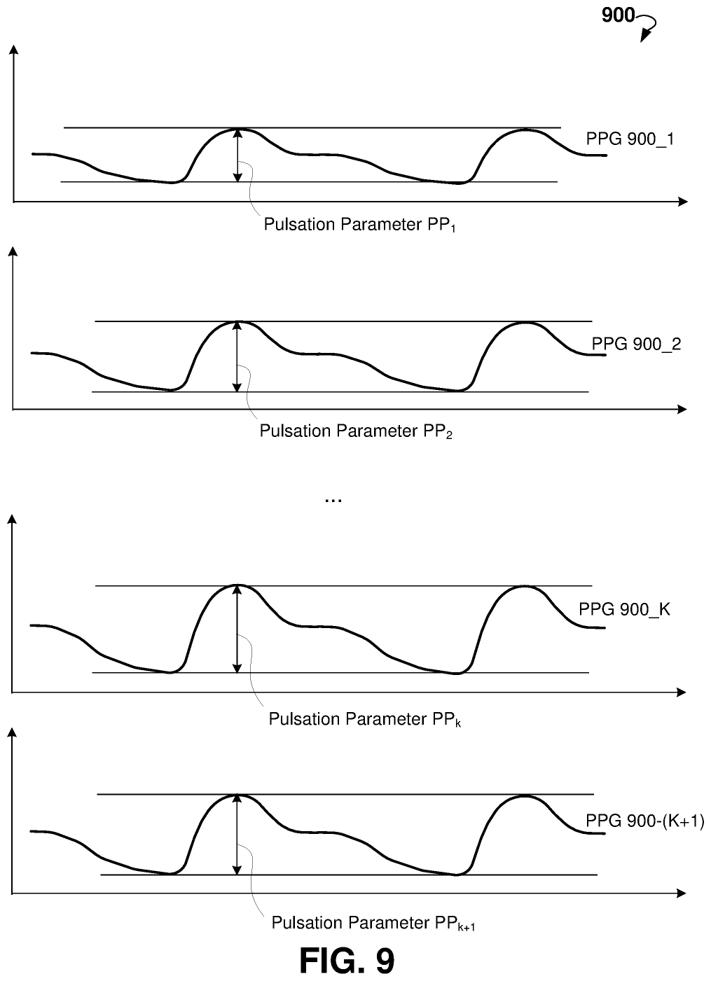

FIG. 10 is a flow chart showing an example method for optimizing sensor pressure in blood pressure measurements, according to some example embodiments. The method 1000 can be implemented using wearable device 110 described with reference to FIGS. 2 and 3 and system 100 described with reference to FIG. 1.

The method 1000 may commence in block 1002 with recording, by at least one processor, PPG) data using a PPG sensor of a wearable device while the pressure applied by the PPG sensor to a blood artery of the user is gradually increasing. The blood artery can be a radial artery of a wrist.

In block 1004, the method 1000 may monitor, by the processor, a pulsating parameter associated with the PPG data. The pulsating parameter may change in response to the gradually increasing pressure and include a difference between a maximum of the PPG data and a minimum of the PPG data.

In block 1006, the method 100 may determine, by the processor, that the pulsating parameter has passed a critical value. The determination that the pulsating parameter has passed the critical value may include determining that the pulsating parameter has stopped increasing and started decreasing. This may indicate that the pulsating parameter has passed the maximum.

The wearable device may include a pressure applying device configured to gradually apply an external pressure to the PPG sensor. Alternatively, the pressure can be increased by the user gradually applying external pressure to the PPG sensor.

In block 1008, in response to the determination that the pulsating parameter has passed the critical value, the method 1000 may stop increasing the pressure. The wearable device may include an alarm unit configured to prompt the user to stop applying the external pressure after the pulsating parameter has passed the critical value. The alarm unit may include a haptic device. The alarm unit may include a sound generating device.

In optional block 1010, the method 1000 may include causing, by processor, a decrease in the pressure to allow the pulsating parameter to return to the maximum. If the pressure is created by the user applying external pressure to the PPG sensor, then the processor may prompt the user to start and stop decreasing the pressure using the alarm unit. If the pressure is created by the pressure applying device, the pressure applying device can decrease the external pressure on the PPG sensor until the pulsating parameter returns to the maximum

In block 1012, the method 1000 may proceed with recording further PPG data using the PPG sensor and electrocardiogram (ECG) data using input plates of the wearable device. In block 1014, the method 1000 may include analyzing, by the processor, further PPG data and ECG data to determine a pulse transit time (PTT), a pulse rate (PR), and a diameter parameter. The diameter parameter may include a change in the diameter of the blood artery.

Determination of the diameter parameter may include modifying the further PPG data by removing, from the further PPG data, an additive contribution resulting from a reflection of a light signal from a surface of a skin covering the blood artery and near-surface tissues underlying the skin and covering the blood artery. During the modification of the PPG data, a contribution resulting from the reflection of the light signal from the blood artery (contribution due to the reflection from the bulk blood volume) is kept unchanged. The additive contribution can be predetermined using a calibration process as described in U.S. patent application Ser. No. 14/738,711, titled “Pulse Oximetry,” filed on Jun. 12, 2015, incorporated herein by reference for all purposes.

The change in the diameter of the blood artery can be determined based on a ratio AC/DC, where AC is an alternating current component of the modified PPG data, and DC is a direct current component of the modified PPG data.

In block 1016, the method 1000 may include determining, by the processor and using a pre-defined model, a BP based on the PTT, the PR, and the diameter parameter. The pre-defined model can establish a relationship between the PTT, the PR, the diameter parameter, and the BP.

Thus, methods and systems for optimizing sensor pressure in blood pressure measurements using wearable devices have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method for optimizing sensor pressure in a blood pressure (BP) measurement, the method comprising:
recording, by at least one processor, photoplethysmogram (PPG) data using a PPG sensor of a wearable device while a pressure applied by the PPG sensor to a blood artery of a user is gradually increasing;
monitoring, by the at least one processor, a pulsating parameter associated with the PPG data, the pulsating parameter changing in response to the gradually increasing pressure;
determining, by the at least one processor, that the pulsating parameter has passed a critical value;
in response to the determination, causing, by the at least one processor, the increase of the pressure to stop;
recording, by the at least one processor, further PPG data using the PPG sensor and electrocardiogram (ECG) data using input plates of the wearable device;
analyzing, by the at least one processor, the further PPG data and the ECG data to determine a pulse transit time (PTT), a pulse rate (PR), and a diameter parameter, wherein the diameter parameter includes a change in the diameter of the blood artery; and
determining, by the at least one processor and using a pre-defined model, a BP based on the PTT, the PR, and the diameter parameter, wherein the pre-defined model establishes a relationship between the PTT, the PR, the diameter parameter, and the BP.

2. The method of claim 1, wherein the wearable device includes a pressure applying device configured to gradually apply an external pressure to the PPG sensor.

3. The method of claim 1, wherein the pressure is increased by the user gradually applying an external pressure to the PPG sensor.

4. The method of claim 3, wherein the wearable device includes an alarm unit configured to prompt the user to stop applying the external pressure after the pulsating parameter has passed the critical value.

5. The method of claim 4, wherein the alarm unit includes a haptic device.

6. The method of claim 4, wherein the alarm unit includes a sound generating device.

7. The method of claim 1, wherein the pulsating parameter is a difference between a maximum of the PPG data and a minimum of the PPG data.

8. The method of claim 1, wherein the determining that the pulsating parameter has passed the critical value includes determining that the pulsating parameter has stopped increasing and started decreasing.

9. The method of claim 8, further comprising, prior to recording the further PPG data, causing, by the at least one processor, a decrease in the pressure to allow the pulsating parameter to return to a maximum.

10. The method of claim 1, wherein:
the determining the diameter parameter includes modifying the further PPG data by removing, from the further PPG data, an additive contribution resulting from a reflection of a light signal from a surface of a skin covering the blood artery and near-surface tissues underlying the skin and covering the blood artery and keeping, in the PPG data, a contribution resulting from the reflection of the light signal from the blood artery unchanged, the additive contribution being predetermined using a calibration process; and
the change in the diameter of the blood artery is determined based on a ratio AC/DC, wherein AC is an alternating current component of the modified PPG data, and DC is a direct current component of the modified PPG data.

11. A system for optimizing sensor pressure in a blood pressure (BP) measurement, the system comprising:
a wearable device including a photoplethysmogram (PPG) sensor and electrocardiogram (ECG) input plates; and
at least one processor communicatively coupled to the wearable device, the at least one processor being configured to:
record PPG data using the PPG sensor while a pressure applied by the PPG sensor to a blood artery of a user is gradually increasing;
monitor a pulsating parameter associated with the PPG data, the pulsating parameter changing in response to the gradually increasing pressure;
determine that the pulsating parameter has passed a critical value;
in response to the determination:
cause stopping the increase of the pressure;
record further PPG data using the PPG sensor and ECG data using the ECG input plates of the wearable device;
analyze the further PPG data and the ECG data to determine a pulse transit time (PTT), a pulse rate (PR), and a diameter parameter, wherein the diameter parameter includes a change in the diameter of the blood artery; and
determine, using a pre-defined model, a BP based on the PTT, the PR, and the diameter parameter, wherein the pre-defined model establishes a relationship between the PTT, the PR, the diameter parameter, and the BP.

12. The system of claim 11, wherein the wearable device includes a pressure applying device configured to gradually apply an external pressure to the PPG sensor.

13. The system of claim 11, wherein the pressure is increased by the user gradually applying an external pressure to the PPG sensor.

14. The system of claim 13, wherein the wearable device includes an alarm unit configured to prompt the user to stop applying the external pressure after the pulsating parameter has passed the critical value.

15. The system of claim 14, wherein the alarm unit includes one of: a haptic device and a sound generating device.

16. The system of claim 11, wherein the pulsating parameter is a difference between a maximum of the PPG data and a minimum of the PPG data.

17. The system of claim 11, wherein the determining that the pulsating parameter has passed the critical value includes determining that the pulsating parameter has stopped increasing and started decreasing.

18. The system of claim 17, wherein prior to recording the further PPG data, the at least one processor causes a decrease in the pressure to allow the pulsating parameter to return to a maximum.

19. The system of claim 11, wherein:
the determining the diameter parameter includes modifying the further PPG data by removing, from the further PPG data, an additive contribution resulting from a reflection of a light signal from a surface of a skin covering the blood artery and near-surface tissues underlying the skin and covering the blood artery and keeping, in the PPG data, a contribution resulting from the reflection of the light signal from the blood artery unchanged, the additive contribution being predetermined using a calibration process; and
the change in the diameter of the blood artery is determined based on a ratio AC/DC, wherein AC is an alternating current component of the modified PPG data, and DC is a direct current component of the modified PPG data.

20. A non-transitory computer-readable storage medium having embodied thereon instructions, which when executed by at least one processor, perform steps of a method, the method comprising:
recording, by at least one processor, photoplethysmogram (PPG) data using a PPG sensor of a wearable device while a pressure applied by the PPG sensor to a blood artery of a user is gradually increasing;
monitoring, by the at least one processor, a pulsating parameter associated with the PPG data, the pulsating parameter changing in response to the gradually increasing pressure;
determining, by the at least one processor, that the pulsating parameter has passed a critical value;
in response to the determination, causing, by the at least one processor, the increase of the pressure to stop;
recording, by the at least one processor, further PPG data using the PPG sensor and electrocardiogram (ECG) data using input plates of the wearable device;
analyzing, by the at least one processor, the further PPG data and the ECG data to determine a pulse transit time (PTT), a pulse rate (PR), and a diameter parameter, wherein the diameter parameter includes a change in the diameter of the blood artery; and
determining, by the at least one processor and using a pre-defined model, a BP based on the PTT, the PR, and the diameter parameter, wherein the pre-defined model establishes a relationship between the PTT, the PR, the diameter parameter, and the BP.
